(19) United States
(12) Patent Application Publication
(10) Pub. No.: US 2020/0000627 A1
(43) Pub. Date: Jan. 2, 2020

(54) DUAL TEMPERATURE, DUAL RESERVOIR CONTRAST PROGRAMMABLE THERAPY DEVICES AND METHODS OF USING THE SAME

(71) Applicant: BOARD OF REGENTS, THE UNIVERSITY OF TEXAS SYSTEM, Austin, TX (US)

(72) Inventors: Kenneth R. DILLER, Elgin, TX (US); Sepideh KHOSHNEVIS, Austin, TX (US); Laura HEMMEN, Lakeway, TX (US); Gary L. MCGREGOR, Pflugerville, TX (US)

(21) Appl. No.: 16/312,534

(22) PCT Filed: Jun. 23, 2017
(86) PCT No.: PCT/US2017/038959

§ 371 Date: Dec. 21, 2018

Related U.S. Application Data
(60) Provisional application No. 62/354,346, filed on Jun. 24, 2016.

(51) Int. Cl. A61F 7/00; A61F 7/02

(52) U.S. Cl. CPC A61F 7/0085; A61F 7/02; A61F 2007/0296; A61F 2007/0093; A61F 2007/0054; A61F 2007/0052

(57) ABSTRACT

Disclosed herein are dual temperature, dual reservoir devices for providing contrast therapy. An example device has separate liquid heating and cooling systems, each of which have their own designated liquid reservoir. In some embodiments, each system is served by its own dedicated energy source, and has its own water flow circuit that is equipped with pressure relief valves and check valves. Some exemplary systems also include electronically controlled three way valves that ensure that either heated liquid or cooled liquid is flowing to and from the therapy pad at any given time, but never simultaneously. This allows for nearly instantaneous switching between the cooling and heating functions. Certain embodiments of the contrast therapy devices disclosed herein also have programmable features that enable a user to adjust certain variables of the therapy, or allow for a personalized therapy regimen to be developed.

### CROSS REFERENCE TO RELATED APPLICATIONS

This application claims the benefit of U.S. Provisional Application No. 62/354,346, filed Jun. 24, 2016, which is hereby incorporated by reference in its entirety for all purposes.

### STATEMENT REGARDING FEDERALLY SPONSORED RESEARCH OR DEVELOPMENT

This invention was made with government support under Grant no. R01 EB015522 awarded by the National Institutes of Health. The government has certain rights in the invention.

### FIELD

The invention generally relates to devices and methods of cryotherapy. More particularly, the invention relates to devices and methods of cryotherapy that combine alternating heating and cooling of injured tissues to minimize ischemic damage to the tissues and to enhance healing processes.

### BACKGROUND

Localized cooling is commonly used to reduce bleeding, inflammation, metabolism, muscle spasm, pain, and swelling following soft tissue trauma and injury. The therapeutic application of cold therapy has a long history dating from the time of Hippocrates and is widely practiced. Over the past two decades the breadth of application has increased dramatically with the advent of mechanized cryotherapy devices consisting of an insulated container filled with an ice/water bath and a submersible pump to propel the flow of ice water through a cooling bladder applied to a therapy site. These devices are now used prescriptively for orthopedic surgical procedures and in many sports and rehabilitation medicine settings. Nonetheless, there remains considerable controversy over the appropriate protocol for application of cryotherapy. One extreme camp advocates continuous use of cryotherapy to a treatment site with no break in cooling for days or even weeks, whereas other practitioners recommend a maximum application duration of 20 to 30 minutes followed by a cessation period of about 2 hours. Many devices and methods are designed and marketed from a perspective that effective and safe cryotherapy depends primarily on regulating the temperature applied to the skin surface, and the duration of cooling is a secondary factor to also be regulated. However, there is a paucity of scientifically derived data that can direct the rigorous and rational design of cryotherapy protocols optimized for therapeutic efficacy and safety. Much of the background understanding that underlies the current practice of cryotherapy is based on anecdotal observations derived from clinical experiences. For example, although continuous cooling appears to be tolerated by many patients, there have been a large number of reported incidences in which continuous application of a cryotherapy device led directly to extensive tissue necrosis and/or nerve injury in the treatment area, sometimes with dire medical consequences.

Although injuries attributed to cryotherapy are frequently classified as frostbite, the fact that cryotherapy units (CTUs) typically use the circulation of melted ice through a flexible pad applied at the treatment site precludes the possibility of actually freezing tissue. Rather, extensive evidence points to tissue damage by nonfreezing cold injury (NFCI) when tissue is subjected to a prolonged state of cold induced vasoconstriction that starves tissues of oxygen and nutrients and allows the accumulation of toxic metabolic byproducts. Therefore, although an applied low temperature is the factor that defines cryotherapy and precipitates both the beneficial and damaging tissue responses, a strong case can be made that the most expedient approach to the design of cryotherapy devices and methods should be guided by considerations of controlling how the perfusion of blood to the treatment area should be manipulated over time.

It is well known that a lowered tissue temperature depresses the conduction velocity in nerves, which, in combination with local ischemia, is thought to relate to the incidence of nerve injury. Cell necrosis may result from a number of complicating factors precipitated by cold-induced ischemia. It has been known for many years that reduced temperatures cause a local decrease in blood perfusion of advantage in treating soft tissue injuries by limiting swelling and inflammation. But, when a prolonged state of ischemia is maintained, cells are deprived of a sufficient supply of nutrients in conjunction with the buildup of metabolic byproducts that, taken together, may lead directly to tissue necrosis and neuropathies. Causation of a prolonged state of ischemia also can lead to the occurrence of reperfusion injury when blood flow is reestablished to the affected tissue. In some cases, these types of injuries are the unfortunate byproduct of the application of cryotherapy. Thus, there is a need to achieve a balance between deriving the benefits of applied cryotherapy while reducing the risk of causing further injury to the tissue being treated, especially when an inherent, concurrent outcome of applying the disclosed invention is an additional improvement in tissue healing.

Avoiding long term ischemia during cryotherapy of extended duration may be achieved by an intermittent, active raising of the tissue temperature to transiently increase perfusion. The alternating cooling and heating of tissue is termed contrast therapy. This concept has been introduced in International Application No. PCT/US2015/038971, which is hereby incorporated by reference in its entirety. For this purpose it is desirable to alternate the skin temperature between lower cooling values and higher warming values. Cooling allows the following therapeutic efficacies to be achieved: (1) to lower blood perfusion for reduced tissue swelling; (2) to lower nerve conduction velocity for reduced pain sensation; (3) to lower the sensitivity of local noxious cold sensors; and (4) to reduce inflammation processes. The short periods of heating allow: (1) elevation of blood flow and metabolic rates to avoid long term ischemia and the potential for tissue injury; (2) prevention of subsequent ischemic reperfusion injury; and (3) improved rates of tissue recovery by exposing the tissue to occasional warm temperatures where healing biochemical processes can proceed at a normal rate.

The informal alternating application of hot and cold packs to injured tissues has long been practiced, but without a rational basis for methodology or a device to ensure accomplishment of targeted therapeutic objectives. The advent some 25 years ago of devices that circulate water from melted ice cubes through a pad placed on a treatment surface initiated wide spread application of the field of cryotherapy. Although simple, these cryotherapy units (CTUs) had limited therapeutic flexibility, being capable of producing only cooling, and that only within a narrow range of temperatures.

Alternative CTUs having superior design and performance have been developed with a thermoelectric chip (TEC) as the source of cooling a reservoir of water that could be circulated through the treatment pad. TECs have multiple advantages over melting ice in that their treatment temperature can be modulated accurately by adjusting the magnitude of the applied electrical voltage. Also, by reversing the polarity of the voltage, the TEC can be changed between functioning as a cooling or a heating source. Thus, in principle, a TEC CTU can be made to provide contrast therapy. However, many limitations still exist. Currently, TEC CTUs are designed with a single energy source that is used for both heating and cooling, and only a single reservoir of water that is circulated through the therapy pad. Although the single TEC may be switched between cooling and heating modes readily, the process requires an added passive period during which control over the therapy process must be forfeited to allow the temperature gradients in the TEC chip to relax back to a neutral status to avoid creating thermal stresses when their direction is reversed by the switch. The TEC chip is fabricated from a brittle material that is subject to fracture by thermal stress. A contrast therapy device is, by definition, required to repeatedly switch between cooling and heating over a very large number of cycles, increasing the likelihood of TEC chip fracture.

Another limitation of existing TEC CTUs is that they have only a single reservoir from which water is circulated to a therapy pad. Thus, when a switch is made between cooling and heating and vice versa, the temperature of the water in the reservoir must be reversed. This process requires added time to alter the water temperature, introducing a further delay in which control of the therapy is compromised, and it is energetically inefficient. A single reservoir system is caught between two compromised situations in addressing a solution to this problem. On the one hand, the volume of water in the reservoir may be made small so that its temperature may be changed between cold and hot relatively easily, but the limited water volume compromises the ability of the system to deliver temperature controlled water to the therapy pad. The result is limited thermal performance and a limited range of thermal therapy protocols that can be produced. Alternatively, the reservoir volume may be increased substantially to provide an adequate convective flow of water to the therapy pad. However, the added water means that both the energy and time required to change its temperature during the switch between heating and cooling modes must also increase substantially, compromising the ability to control the temperature/time history and the thermal efficiency of the device.

### SUMMARY

Disclosed herein are dual temperature, dual reservoir devices for providing programmable contrast therapy. An example device has separate liquid heating and cooling systems, each of which have their own designated liquid reservoir. Each of the heated and cooled liquid reservoirs contain a volume of water adequate to provide effective thermal therapy via flow through a single therapy pad. In some embodiments, each system is served by its own dedicated energy source, and has its own water flow circuit that is equipped with pressure relief valves and check valves. Some exemplary systems also include electronically controlled three way valves that ensure that either heated liquid or cooled liquid is flowing to and from the therapy pad at any given time, but never simultaneously. This allows for nearly instantaneous switching between the cooling and heating functions. Certain embodiments of the contrast therapy devices disclosed herein also optimally have programmable features that enable a user to adjust certain variables of the therapy, or allow for a personalized therapy regimen to be developed. The described methods are designed to prevent the development of persistent ischemia in the treatment area.

### DESCRIPTION OF DRAWINGS

FIG. 1A shows a cross sectional view of a liquid-perfused pad for contrast therapy.

FIG. 1B shows a top down view of a liquid-perfused pad for contrast therapy.

FIG. 2A shows a flow schematic of an embodiment of a contrast therapy device.

FIG. 2B shows a flow schematic of an embodiment of a contrast therapy device.

FIG. 3 shows a graph of the temperature and perfusion of the skin during a contrast therapy regimen using a contrast therapy device.

FIG. 4 shows the water temperature at the inlets and outlets of the liquid perfused pad for an embodiment of the disclosed contrast therapy device as compared to a competing device.

FIG. 5 shows the temperature at the skin of a subject being treated with an embodiment of the disclosed contrast therapy device as compared to a competing device.

FIG. 6 is a graph comparing the temperature at the skin surface and three centimeters into the underlying muscle tissue undergoing contrast therapy.

FIG. 7 shows the temperature at the surface of a pad of an embodiment of the contrast therapy device.

### DETAILED DESCRIPTION

The following description of certain examples of the inventive concepts should not be used to limit the scope of the claims. Other examples, features, aspects, embodiments, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the embodiments of this disclosure are described herein. The described methods, systems, and apparatus should not be construed as limiting in any way. Instead, the present disclosure is directed toward all novel and nonobvious features and aspects of the various disclosed embodiments, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect, embodiment or example of the invention are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

Disclosed herein are dual temperature, dual reservoir devices for providing contrast therapy. An example device has separate liquid heating and cooling systems, each of which have their own designated liquid reservoir. Each of the heated and cooled liquid reservoirs contain a volume of water adequate to provide effective thermal therapy via flow through a single therapy pad. In some embodiments, each system is served by its own dedicated energy source for cooling and heating, and has its own water flow circuit that is equipped with pressure relief valves and check valves. Some exemplary systems also include electronically controlled three way valves that ensure that either heated liquid or cooled liquid is flowing to and from the therapy pad at any given time, but never simultaneously. This allows for nearly instantaneous switching between the cooling and heating functions. Certain embodiments of the contrast therapy devices disclosed herein also have programmable features that enable a user to adjust certain variables of the therapy, or allow for a personalized therapy regimen to be developed based on a patient's unique anatomical structure and/or therapeutic requirements.

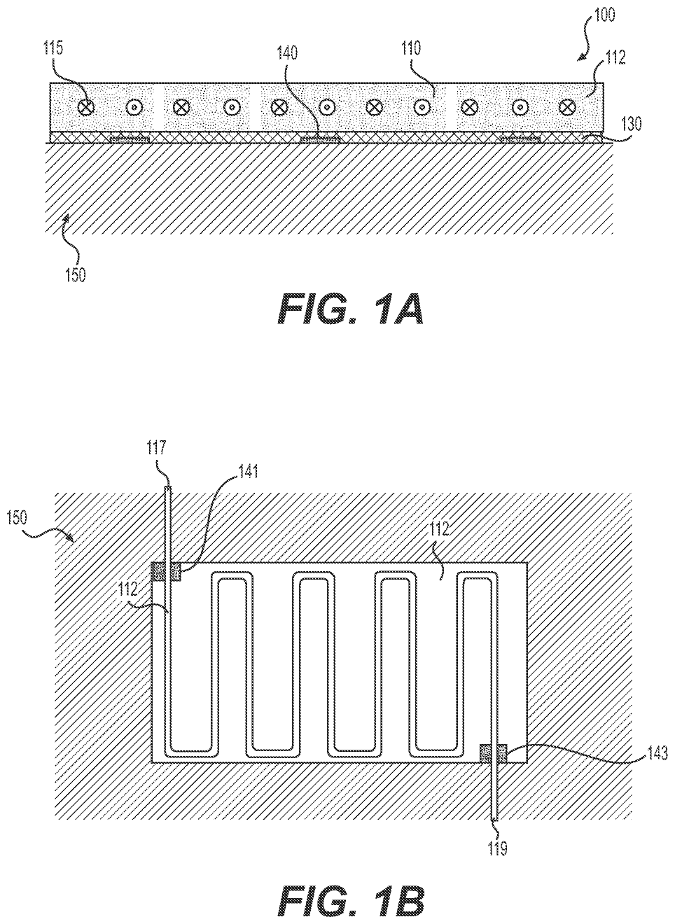

FIGS. 1A-B depict the liquid perfused pad 110 of a contrast therapy device 100. A thermal barrier 130 is arranged between the liquid perfused pad 110 and the tissue 150 as depicted in FIG. 1A. Liquid-perfused pad 110 includes a support medium 112 and a serpentine conduit 115 which extends throughout the support medium. Serpentine conduit 115 includes an inlet path 117 and an outlet path 119 which transmit cooling fluid from an external source of cooling fluid (not shown) through the serpentine conduit. One or more tissue temperature sensors 140 are used for feedback to a programmable central processing module. For example, a first temperature sensor 141 can be positioned at or near the inlet path 117, and a second temperature sensor 143 can be positioned at or near the outlet path 119. The device shown is incorporated into a single unit that is applied at a site of therapy. The device is positionable on the tissue 150 of a subject. The liquid used in the embodiments disclosed herein is a non-gaseous liquid. In some embodiments, the liquid is water. Some embodiments of the system can also include a disposable cover configured to fit over or be attached to the liquid perfused pad.

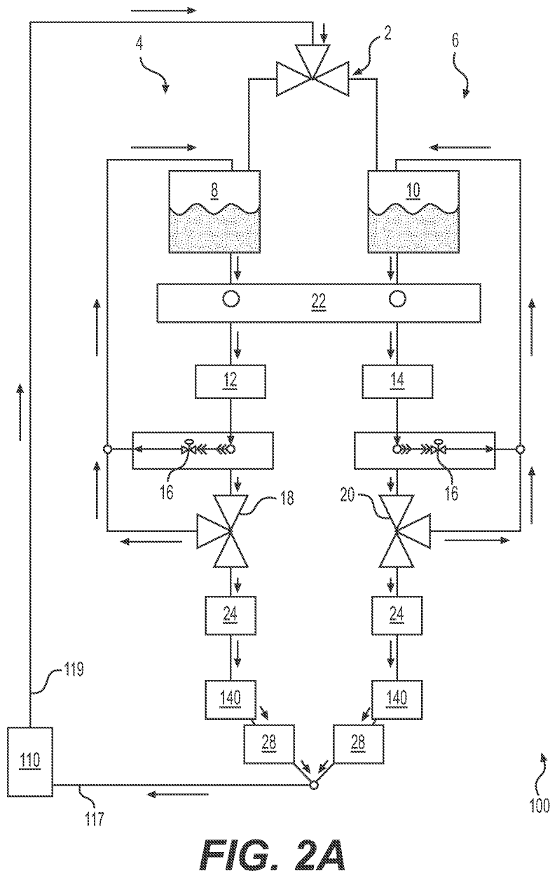

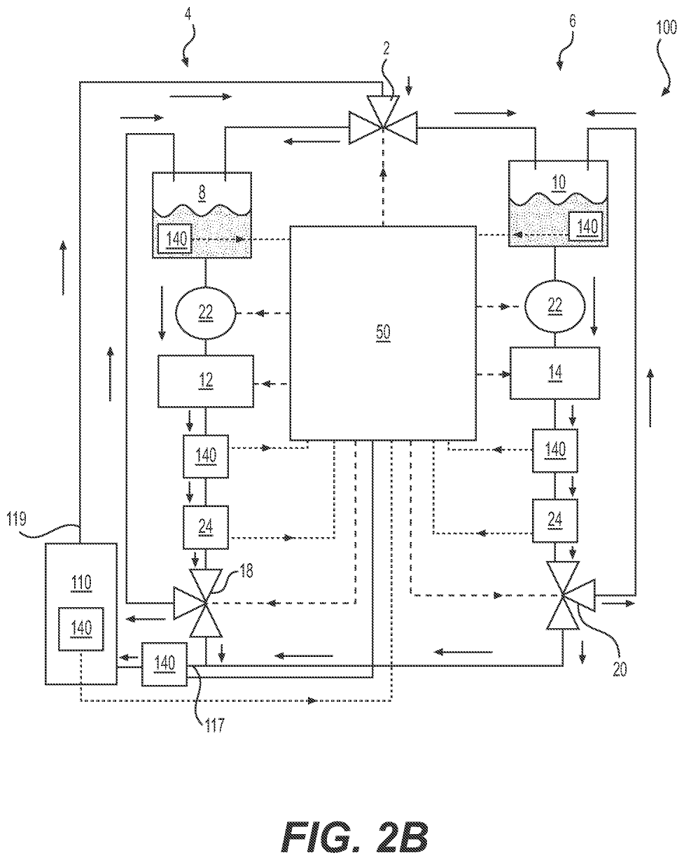

FIGS. 2A and 2B show a flow diagrams of two example embodiments of contrast therapy devices 100. The liquid flows from the inlet path 117 through the liquid-perfused pad 110 and out the outlet path 119. At the end of the outlet path 119, a first valve 2 controls the routing of the liquid into either the liquid heating system 4 or the liquid cooling system 6, depending on the current state of the contrast therapy program. In some embodiments, the first valve 2 is a three-way solenoid valve. However, the first valve 2 may also be any valve for which the inlet flow may be directed selectively to any of multiple outlets.

After passing through the first valve 2, the liquid in the embodiments shown in FIGS. 2A and 2B enters either the heated liquid reservoir 8 or the cooled liquid reservoir 10. The temperature of the liquid in each reservoir is maintained by separate heating and cooling elements 12, 14. In some embodiments, the heating element 12 operates via dissipation of electromagnetic energy. The heating element 12 could be, for example, a resistive heating element (using ohmic heating), an inductive heating element, or an element that uses any dissipative energy generation mechanism. The cooling element 14 can be, in some embodiments, a thermoelectric chip (TEC).

As shown in FIG. 2A, some embodiments can include a mechanical pressure relief valve 16 positioned within the heating system 4 or within the cooling system 6 (or both), to prevent leakage should the pressure in the system exceed a certain range. The mechanical pressure relief valve 16 may empty back into the reservoir, or out of the system entirely.

A heated liquid outlet valve 18 or cooled liquid outlet valve 20 can be placed downstream of the heating/cooling systems, as shown in FIGS. 2A and 2B. The liquid outlet valves 18, 20 can be three way solenoid valves. However, the liquid outlet valves 18, 20 can also be any valve for which the flow may be directed selectively to any of multiple outlets. The heated liquid outlet valve 18 routes the heated liquid back toward the liquid-perfused pad 110 (when open) or back into the heated liquid reservoir 8 (when closed). If the heated liquid outlet valve 18 is in the closed position, the mechanical pressure relief valve 16 may also serve to route the heated liquid back into the reservoir 8. Likewise, for the cooling system 6, the cooled liquid outlet valve 20 routes the cooled liquid back toward the liquid-perfused pad 110 (when open) or back into the cooled liquid reservoir 8 (when closed). The heated liquid outlet valve 18 and the cooled liquid outlet valve 20 operate in synchrony such that either heated or cooled liquid is flowing toward the liquid-perfused pad 110, but never both. This enables the liquid-perfused pad 110 to undergo rapid changes in temperature without requiring a lengthy warm up or cool down period.

As shown in FIGS. 2A and 2B, the liquid within each system 4, 6, is kept in motion by one or more pumping units 22. The pumping unit may be a single-motor pumping unit with two parallel pumps, one for controlling the heating system 4 and one for controlling the cooling system 6, as shown in FIG. 2A. Alternatively, there can be two separate pumping units with two separate motors for controlling the individual systems, as shown in FIG. 2B.

In some embodiments, such as the ones shown in FIGS. 2A and 2B, pressure transducers 24 may be positioned along the flow lines. The pressure transducers 24 shown in FIG. 2A are positioned downstream of the liquid outlet valves 18, 20, but pressure transducers could be positioned at other locations (for example, along the flow lines of the heating system 4, the cooling system 6, or along the inlet or outlet paths 117, 119). For example, in the example shown in FIG. 2B, the pressure transducers 24 are positioned upstream of the liquid outlet valves 18, 20. The pressure transducers 24 monitor the pressure in the system to detect pressures that are too high or too low. The pressure transducer 24 can be designed to alter the volume flow rate of liquid from the pump, activate a pressure relief valve, or to shut down the system upon detection of an abnormally high pressure. A pressure that is abnormally low may indicate the presence of a leak.

As shown in FIGS. 2A and 2B, temperature sensors 140 may be placed throughout the contrast therapy device 100. Particularly, it can be advantageous to place sensors 140 at points on or near the liquid-perfused pad 110, as shown in FIG. 2B. For example, a temperature sensor 140 may be located at or near the inlet path 117, as shown in FIGS. 2A and 2B. Alternatively, temperature sensors may be placed at the interface of the pad 110 and the tissue 150, or along the outlet path 119. Other locations that can include temperature sensors 140 include any point within the heating or cooling systems 4, 6, (for example, to take the temperature of the liquid in the reservoirs 8, 10, as shown in FIG. 2B) or any point downstream of the outlet valves 18, 20 but upstream of the inlet path 117. The output of one or more temperature sensors may be applied as a feedback to modulate the function of the heating and/or cooling units.

Flow rate sensors may also be included as part of a contrast therapy device 100. The flow rate sensors detect the speed of the flow of the liquid through the lines of the systems. The flow rate sensors could be located anywhere within the contrast therapy device. They may be particularly advantageous when placed along the inlet and/or outlet paths 117, 119, or within the liquid perfused pad 110.

Some embodiments of contrast therapy devices 100 may include check valves 28 to prevent backflow of liquid within the flow lines. These are especially important near the inlet path 117, as shown in FIG. 2, to prevent mixing of heated and cooled liquid between the heating and cooling systems 4, 6. The check valves 28 may be placed at any point within the contrast therapy device, however, including within the heating system 4 and/or within the cooling system 6, and/or downstream of the outlet valves 18, 20.

As shown in FIG. 2B, the contrast therapy devices 100 disclosed herein can also be equipped with a central processing module 50 programmed to adhere to an automated heating and cooling program, and/or to adapt to measurements from a patient to provide a more personalized therapy. The central processing module 50 can act as a controller, a logger, and/or a reader. The logging functionality of the central processing module 50 can, for example, record the temperature history, and the reading functionality can, for example, read sensor signals and accept input from a user, as described below. FIG. 2B shows the direction of control signals as long-dashed lines, the direction of sensor signals as short-dashed lines, and the direction of water flow as black lines for this exemplary embodiment. The control functionalities are described in greater detail, below.

The central processing module 50 can be configured to perform one or more of the following functions: control the heating and cooling elements and the temperature(s) of the hot and/or cold reservoirs, control the direction of flow from valves 2, 16, 18, and 20, control delivery of either heated or cooled liquid via control of the heated and cooled liquid outlet valves, control the pump rate of water circulating through the systems, control the volume of water in the reservoirs 8, 10, record data from one or more temperature sensors, record data from one or more flow rate sensors, detect deviation of temperatures from preset ranges, detect deviation of flow rate from preset range, detect deviation of pressure from preset range, alert the user if a deviation is detected, download data recorded during a therapy protocol, and shut down the device if a deviation outside a present range is detected.

The contrast therapy device may also be equipped with a user interface to receive input from a user. The central processing module 50 can be configured to execute inputs from the user interface. In some embodiments, the user interface can be used to enable a user to directly control certain variables of the contrast therapy device 100. The variables include but are not limited to: temperature of liquid in the heated liquid reservoir, temperature of liquid in the cooled liquid reservoir, flow rate of liquid through the liquid-perfused pad, and the timing of release of heated or cooled liquid into the inlet path. The user interface can include a display that informs the user of key operating states, such as temperatures and flow rate.

In some embodiments, the contrast therapy device 100 is equipped to provide the subject with personalized therapy. The central processing module 50 can be configured to receive one or more physiological, medical, or anatomical measurements of a subject and to calculate and perform a personalized thermal therapy treatment on the subject based on the measurements. The measurements can, in some embodiments, be sourced from sensors that are operatively connected to the subject, or in some embodiments, the measurements can be entered into the user interface. The measurements can then be used by the central processing module 50 to calculate and augment certain variables of the treatment regiment, including but not limited to: temperature of liquid in the heated liquid reservoir, temperature of liquid in the cooled liquid reservoir, flow rate of liquid through the liquid-perfused pad, and the timing of release of heated or cooled liquid into the inlet path.

The contrast therapy device 100 is used to deliver thermal contrast treatment to the tissue 150 of a subject. The treatment may be used to enhance the healing process for an area of injured soft tissue or to precondition tissue prior to an anticipated trauma such as may occur during a surgical procedure or during participation in a stressful physical activity such as an athletic competition. The pad 110 is first applied to the body surface requiring the treatment. Next, either heated or cooled liquid is run from the respective reservoir 8, 10 to the liquid perfused pad 110. There is no mixing of the heated and cooled liquid due to the aforementioned setup. The liquid runs from the respective reservoir, into the inlet path of the pad 117, through the pad, and out the outlet path 119 of the pad during a particular cooling or warming period.

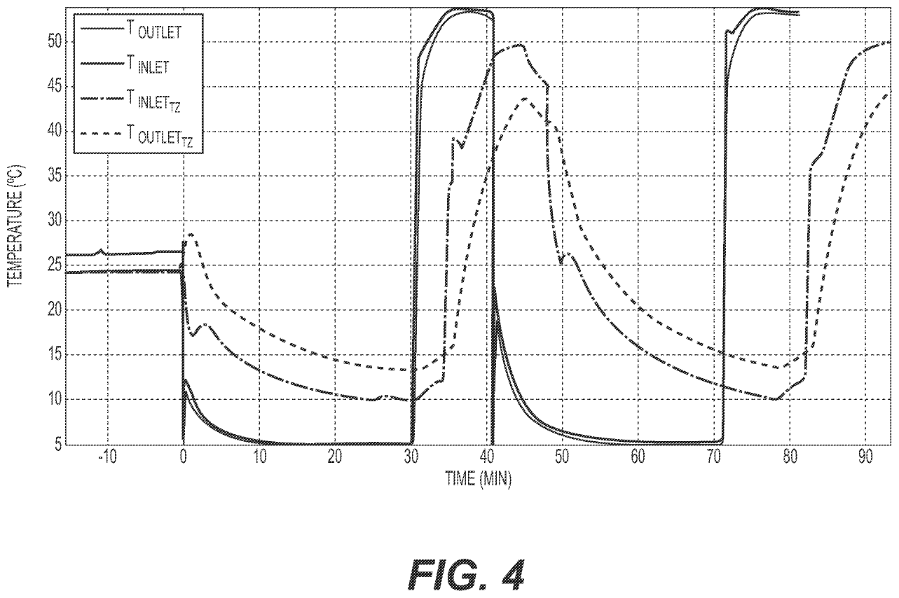

The transition period between cooling and warming periods is very brief, due to the aforementioned setup that enables rapid shutoff of the liquid flow from one system and rapid initiation of flow from the other system. The switching time between water flows from the warm and cool reservoirs can be from 1 to 6 seconds, for example, about 3 seconds, or the time it takes for the water to complete a circulation loop through the whole system. During the switching time, the pump or pumps can be turned off simultaneously to avoid over pressurizing the system. In fact, temperature sensors 140 positioned at the outlet 119 of the liquid perfused pad 110 can register a temperature change over the range of anywhere from 5 to 50 degrees Celsius over the course of a 1 minute transition period, as shown in FIG. 4. In some cases, the transition period yielding temperature changes of from 5 to 50 degrees Celsius may be as brief as 15 seconds. Interestingly, the heated liquid can reach a maximum of greater than 50 degrees Celsius as measured at the outlet 119, and the cooled liquid can reach a minimum of less than 10 degrees Celsius as measured at the outlet. However, if pad 110 is applied directly to the body, the maximum temperature of the heated liquid should not exceed 43 degrees Celsius. The difference in temperature of liquid measured at the inlet path 117 versus the outlet path 119 never exceeds 5 degrees Celsius.

Therapeutic variables such as the temperature of the liquid, the flow rate of the liquid, and the duration of the cooling or warming period may be set to an automated program, or may be controlled at any point during the therapy by a user via the user interface. As used herein, a “user” may be a healthcare practitioner.

Alternatively or in conjunction, the therapeutic variables may be controlled via feedback from the subject, so as to deliver a personalized therapy. The feedback may come from the subject in the form of physiological, medical or anatomical measurements from sensors operatively connected to the subject, or from the subject entering instructions into the user interface. As used herein, a “subject” is the living being receiving the contrast therapy treatment. The method can then include setting one or more variables of the contrast therapy including the temperature of the heated liquid, the temperature of the cooled liquid, the duration of the heating period, or the duration of the warming period based on the physiological, medical, or anatomical measurements or instructions from or about the subject. For example, the heat transfer properties of a tissue are greatly dependent on the size of the subject; it would take less time for heat to reach the ACL of a child's knee than an adult's knee. Anatomical measurements can be used to set the duration of the heating cycle, and physiological measurements (temperature at the skin surface, for example) can provide feedback to the system, at which point the central processing module could switch to a cooling mode in the event that the area is overheating.

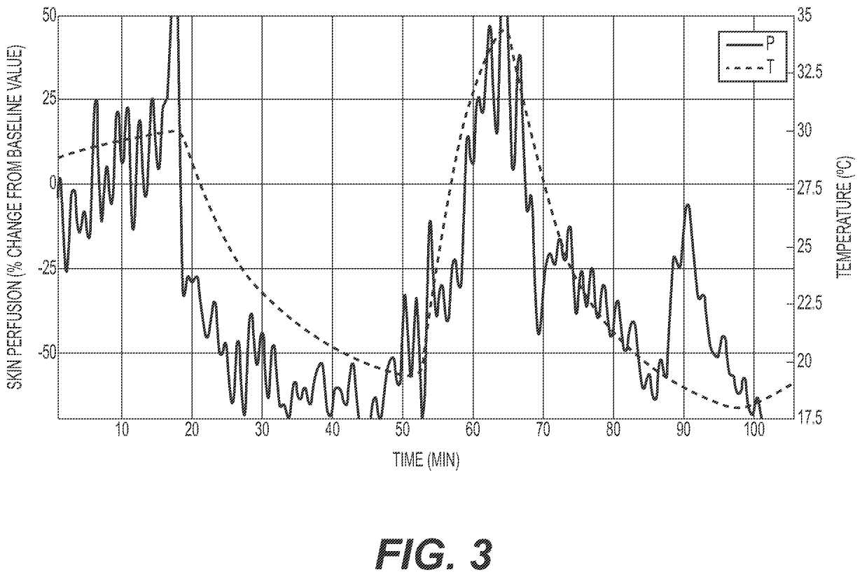

FIG. 3 shows the temperature on the skin surface and blood flow in the target tissue for an example contrast therapy cycle. The contrast therapy cycle shown consists of an initial baseline followed by cooling for 33 minutes, warming for 14 minutes, and cooling for 33 minutes. The use of contrast therapy with rapid cooling and warming results in the blood perfusion in the treatment area following the trends of the temperature application on the skin, which is especially important during warming. Passive rewarming by heat gained only from the environment can result in hours of exposure after completion of a cooling episode during which the blood flow is deeply depressed, which may lead to ischemic nonfreezing cold injury to tissue and nerves. In the exemplar trial under the action of active warming the blood flow is returned to the baseline value within only eight minutes.

FIG. 4 shows the temperature plots for the inlet and outlet water temperatures for a standard commercial serpentine flow pattern therapy pad having a nominal ¼ inch channel diameter. The temperature sensors can be configured such as is shown in FIG. 1B, with a first temperature sensor 141 positioned at or near the inlet 117, and a second temperature sensor 143 positioned at or near the outlet 119. Identical contrast protocols were programmed using an embodiment of the dual temperature dual reservoir (DTDR) system disclosed herein and a current commercial TEC CTU (ThermaZone by Innovative Medical Equipment), which is a single small-reservoir system. The protocol consisted of a contrast cycle consisting of an initial baseline followed by cooling for 30 minutes, warming for 12 minutes, and cooling for 30 minutes. The solid lines are for performance of a DTDR prototype system. Each dual temperature reservoir contains about 1 liter of water for circulation to the therapy pad. The dashed lines are for a TEC CTU (ThermaZone) that has a small reservoir volume (about 60 ml).

As demonstrated by FIG. 4, there are several operational features that distinguish the performance of the DTDR system from the existing technology. First, the response times for causing cooling and warming are much more rapid for the DTDR system than for the small volume TEC CTU system. The TEC CTU device does not have an adequate flow of controlled temperature water to provide convective heat transfer as it flows through the therapy pad to be able to operate without a large time constant lag in thermal performance This condition of low flow with a large time lag is a major limiting factor in the ability to execute a contrast therapy protocol on a patient as may be prescribed by a health care provider. Second, the temperature difference between the inlet and outlet temperatures of the water flowing through the therapy pad is about ten times larger for the TEC CTU system than for the DTDR system. This difference is a consequence of the fact that the heat removed from a smaller volume of water, at a necessarily slower flow rate, will cause a larger change in temperature than for a larger volume of flowing water. The large temperature difference as the water from the TEC CTU source flows through the therapy pad means that there must be an inherently greater variation in the applied therapy conditions than is accomplished with the DTDR system. The inlet and outlet water temperature difference for the DTDR system is inconsequentially small, meaning that essentially uniform therapy conditions may be created on the pad surface. Third, the minimum cooling temperature and maximum heating temperatures are much more extreme for the DTDR system than for the TEC CTU system. In practice, this performance differential means that the prescribed conditions of a contrast therapy protocol will be more accurately achieved with the DTDR system, providing better quality control of the patient treatment. For example, for the warming aspect of the contrast therapy cycle, the DTDR maintains approximately a 30° C. temperature differential from baseline, and that value is reached nearly immediately following the start of warming. The TEC CTU system reaches an average of only about a 20° C. temperature differential from baseline, a drop in performance by a factor of ⅓. Plus, the time lag shows that about ¾ of the entire heating period is required to achieve the maximum temperature. Combining these features of the weaker thermal performance of the TEC CTU mean that the quality of a contrast therapy protocol would be greatly compromised. Finally, the DTDR system is able to sustain a constant minimum cooling temperature and maximum warming temperature for nearly the entirety of the programed periods during the contrast therapy protocol. The weaker performance of the TEC CTU results in an essentially continuously varying temperature delivered to the therapy pad. The precision of the TEC CTU suffers greatly in comparison with the DTDR system.

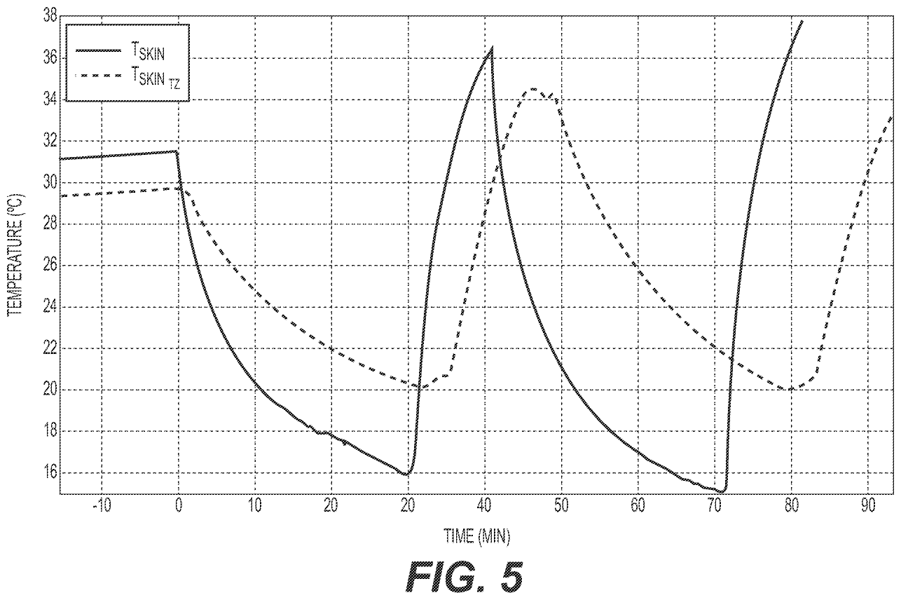

FIG. 5 shows the results of an additional trial conducted to compare the actual contrast therapy temperatures that are produced at a treatment site for a human subject with the DTDR and TEC CTU systems for identical programmed temperature/time protocols. The data from this trial represents the actual contrast therapy protocol that would be produced on a patient by the DTDR and TEC CTU systems. The rates of cooling and warming are much greater for the DTDR system than for the TEC CTU system. It is well documented that more rapid temperature changes on the skin surface produce an improved control of the blood perfusion response in tissue as well as providing extended periods of true therapeutic benefit. Also, the minimum cooling temperature and maximum warming temperatures achieved with the DTDR system have substantially larger differentials from the baseline value than for the TEC CTU system. These temperatures differentials are on the order of 40%-50% greater for the DTDR system, once again demonstrating its improved performance over the TEC CTU system.

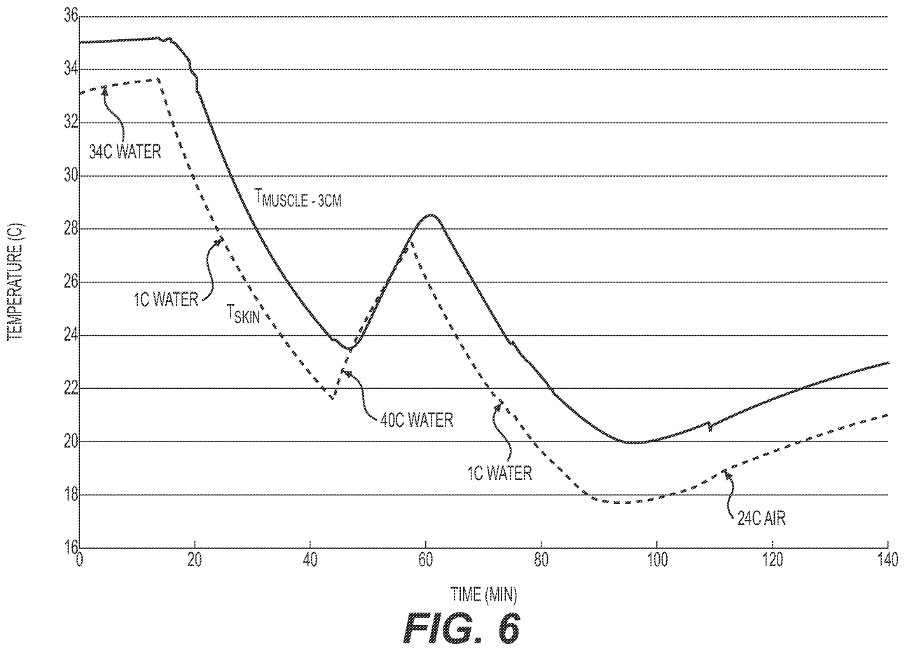

FIG. 6 show results from a trial focusing on the effects of the DTDR system within deep muscle tissue. Temperatures were measured on the skin surface and in calf muscle 3 centimeters deep during a contrast therapy protocol using a rigid thermocouple probe inserted to target depth. Pad 110 was applied to the lateral calf. Temperature of the circulating water was initially 34 degrees Celsius, followed by a 30 minute period of circulation of cooling water at 1 degree Celsius, followed by a 12 minute period of circulation of water at 40 degrees Celsius for active contrast warming, followed by a second 30 minute period of circulation of cooling water at 1 degree Celsius, followed by exposure to room temperature air at 24 degrees Celsius for passive warming. FIG. 4 demonstrates that alternating surface cooling and heating reaches to deep muscle. A temperature drop during cooling of 11 degrees Celsius is achieved within the deep muscle after 30 minutes of cooling followed by a rise of 5 degrees Celsius with 12 minutes of active contrast warming. A second cooling episode further reduces the deep muscle temperature by 15 degrees Celsius. The deep tissue thermal response lags the surface by a time constant of about 3 minutes, and the deep temperatures are continuously warmer than the surface by about 2 degrees Celsius, except during active contrast warming (owing to combined metabolic, blood perfusion and heat diffusion effects). A 48 minute cooling protocol will produce even lower temperatures that are well within the range for pain reduction and reduced perfusion (and swelling). Prior studies have suggested that it is not possible to affect the temperature of deep muscle tissue by contrast therapy. However, it must be realized such studies were constrained by restricting the cooling period to 1 minute, which is not long enough to produce a deep muscle tissue thermal effect. Other studies have used ice and cold packs for 30 and 20 minutes to produce muscle temperature drops 3 centimeters deep, but without the benefits of the warming periods offered by contrast therapy.

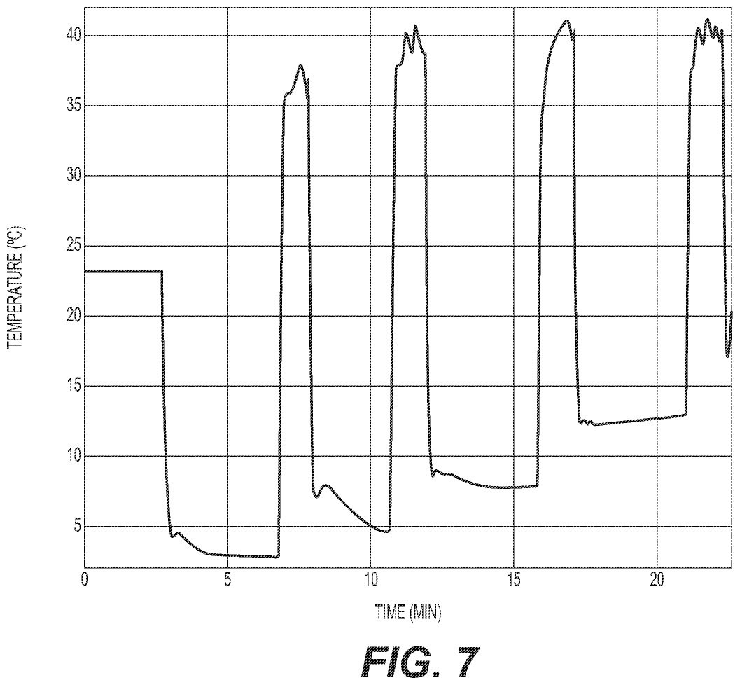

FIG. 7 shows a temperature history taken at a pad 110 of an embodiment of the contrast therapy device 100. The rapid temperature changes seen between the 4 minute cooling and 1 minute heating periods demonstrate a high degree of control over the temperature applied to the subject, which is not possible to achieve by conventional techniques including single reservoir thermoelectric systems.

1. A contrast therapy device for alternating the application of cooler and warmer temperatures to a body surface, the contrast therapy system comprising;
a liquid-perfused pad for application to a body surface,
an inlet path for routing liquid into the liquid-perfused pad,
an outlet path for routing liquid out of the liquid-perfused pad,
a liquid heating system comprising a heated liquid reservoir, a heating element, and a heated liquid outlet valve configured to control release of heated liquid into the inlet path
a liquid cooling system comprising a cooled liquid reservoir, a cooling element, and a cooled liquid outlet valve configured to control release of liquid into the inlet path, and
a first valve fluidly connected to the outlet path and configured to route liquid from the outlet path into either the liquid heating system or the liquid cooling system.

2. The contrast therapy device of claim 1, wherein the device comprises at least one flow rate sensor, wherein the flow rate sensor is operatively connected to at least one of the liquid-perfused pad, the inlet path, and the outlet path.

3. The contrast therapy device of claim 1, wherein the device comprises at least one pressure transducer operatively connected to at least one of the liquid heating system or the liquid cooling system; wherein the at least one pressure transducer is operatively connected to the liquid heating system at a point between the heated liquid outlet valve and the inlet path, and/or the at least one pressure transducer is operatively connected to the liquid cooling system at a point between the cooled liquid outlet valve and the inlet path.

4. (canceled)

5. (canceled)

6. The contrast therapy device of claim 1, wherein the device comprises at least one mechanical pressure relief valve operatively connected to at least one of the liquid heating system and the liquid cooling system, wherein the at least one mechanical pressure relief valve is operatively connected to the liquid heating system at a point between the heating element and the heated liquid outlet valve, and/or the at least one mechanical pressure relief valve is operatively connected to the liquid cooling system at a point between the cooling element and the cooled liquid outlet valve.

7. (canceled)

8. (canceled)

9. The contrast therapy device of claim 1, wherein the device comprises at least one check valve operatively connected to at least one of the liquid heating system and the liquid cooling system, wherein the at least one check valve is operatively connected to the liquid heating system at a point between the heated liquid outlet valve and the inlet path, and/or the at least one check valve is operatively connected to the liquid cooling system at a point between the cooled liquid outlet valve and the inlet path.

10. (canceled)

11. (canceled)

12. The contrast therapy device of claim 1, wherein the device comprises one or more pumping units, wherein at least one pumping unit is a single pumping unit comprising a single motor, a first parallel pump configured to pump the heated liquid, and a second parallel pump configured to pump the cooled liquid.

13. (canceled)

14. The contrast therapy device of claim 1, wherein the device comprises a user interface, wherein the user interface is configured to allow a user to control one or more of the following variables of the system: temperature of liquid in the heated liquid reservoir, temperature of liquid in the cooled liquid reservoir, flow rate of liquid through the liquid-perfused pad, and the timing of release of heated or cooled liquid into the inlet path.

15. The contrast therapy device of claim 1,-wherein the device comprises a central processing module configured to perform one or more of the following functions: execute inputs from a user interface, control the temperatures of the liquid in the heated liquid reservoir and of the liquid in the cooled liquid reservoir, control delivery of either heated or cooled liquid via control of the heated and cooled liquid outlet valves, record data from one or more temperature sensors, record data from one or more flow rate sensors, detect deviation of temperatures from preset ranges, detect deviation of flow rate from preset range, detect deviation of pressure from preset range, alert the user if a deviation is detected, download data recorded during a therapy protocol, shut down the device if a deviation outside a present range is detected, and receive the one or more physiological, medical, or anatomical measurements via a user interface, via sensors operatively connected to the subject, or both.

16. The contrast therapy device of claim 15, wherein the central processing module of the device is configured to receive one or more physiological, medical, or anatomical measurements of a subject and to calculate and perform a personalized thermal therapy treatment on the subject based on the one or more physiological, medical, or anatomical measurements.

17. The contrast therapy device of claim 16, wherein the measurements are used to determine one or more of the following variables of the system: temperature of liquid in the heated liquid reservoir, temperature of liquid in the cooled liquid reservoir, flow rate of liquid through the liquid-perfused pad, and the timing of release of heated or cooled liquid into the inlet path.

18. (canceled)

19. The contrast therapy device of claim 1, wherein the first valve is a 3 way solenoid valve

20. The contrast therapy device of claim 1, wherein the cooling element is a thermoelectric chip (TEC).

21. The contrast therapy device of claim 1, wherein the heating element operates via dissipation of electromagnetic energy.

22. (canceled)

23. (canceled)

24. The contrast therapy device of claim 1, wherein the device comprises at least one temperature sensor operatively connected to at least one of the liquid heating system and/or the liquid cooling system.

25. (canceled)

26. The contrast therapy device of claim 1, wherein the liquid-perfused pad comprises a temperature sensor.

27. The contrast therapy device of claim 1, wherein the heated liquid outlet valve is configured to route release of heated liquid into either the inlet path or back into the liquid heating system.

28. The contrast therapy device of claim 27, wherein the heated liquid outlet valve is a 3 way solenoid valve.

29. The contrast therapy device of claim 1, wherein the cooled liquid outlet valve is configured to route release of cooled liquid into either the inlet path or back into the liquid cooling system.

30. The contrast therapy device of claim 29, wherein the cooled liquid outlet valve is a 3 way solenoid valve.

31-33. (canceled)

34. A method of applying contrast therapy to a patient, the method comprising
applying a pad to a body surface,
running heated liquid from a heated liquid reservoir into an inlet path of a pad, through the pad, and out the outlet path of the pad during a warming period,
running cooled liquid from a cooled liquid reservoir into an inlet path of a pad, through the pad, and out the outlet path of the pad during a cooling period, and
switching from the warming period to the cooling period, or from the cooling period to the warming period, over a transition period.

35. The method of claim 34, wherein the heated liquid reaches a maximum of greater than 43 degrees Celsius as measured at the outlet.

36. The method of claim 34, wherein the cooled liquid reaches a minimum of less than 10 degrees Celsius as measured at the outlet.

37. The method of claim 34, wherein the difference in temperature of liquid measured at the inlet versus the outlet never exceeds 5 degrees Celsius.

38. (canceled)

39. The method of claim 34, further comprising generating one or more physiological, medical or anatomical measurements of a subject and setting one or more variables of the contrast therapy including the temperature of the heated liquid, the temperature of the cooled liquid, the duration of the heating period, or the duration of the warming period based on the physiological, medical, or anatomical measurement.

40. The method of claim 34, wherein the temperature difference between heated liquid during the warming period and cooled liquid during the cooling period is from 5 to 43 degrees Celsius as measured at the outlet.

41. The method of claim 34, wherein the duration of the transition period is less than 1 minute.
